(19) United States
(12) Patent Application Publication
(10) Pub. No.: US 2019/0365285 A1
(43) Pub. Date: Dec. 5, 2019

(54) BIOLOGICAL INFORMATION ACQUISITION DEVICE

(71) Applicant: DAIKIN INDUSTRIES, LTD., Osaka-shi, Osaka (JP)

(72) Inventors: Hiroko Kashimoto, Osaka-shi, Osaka (JP); Chika Koyama, Osaka-shi, Osaka (JP); Mamoru Okumoto, Osaka-shi, Osaka (JP); Takehiko Hiei, Osaka-shi, Osaka (JP)

(73) Assignee: DAIKIN INDUSTRIES, LTD., Osaka-shi, Osaka (JP)

(21) Appl. No.: 16/485,672

(22) PCT Filed: Feb. 16, 2018
(86) PCT No.: PCT/JP2018/005512

§ 371 Date: Aug. 13, 2019

(30) Foreign Application Priority Data
Feb. 22, 2017 (JP) 2017-031056

(51) Int. Cl. A61B 5/11; A61B 5/113; A61B 5/00; A61B 5/0205

(52) U.S. Cl. CPC A61B 5/11; A61B 5/113; A61B 5/0816; A61B 5/0205; A61B 2560/0214; A61B 5/6804

(57) ABSTRACT

A biological information acquisition device includes a body motion detector that includes a pressure sensitive portion on which a body motion of a subject acts and that detects a signal indicating the body motion of the subject, and a clamp member that clamps a clothing item of the subject. The clamp member includes a first member that is disposed on the subject side of the clothing item, and a second member that is disposed on the opposite side of the clothing item from the first member. The pressure sensitive portion is disposed on the first member side.

### TECHNICAL FIELD

The present invention relates to a biological information acquisition device that is wearable by a subject.

### BACKGROUND ART

There are biological information acquisition devices that acquire biological information of a subject.

PTL 1 describes a belt-type biological information acquisition device, which is an example of such devices. As illustrated in, for example, FIG. 2 of PTL 1, the biological information acquisition device includes a belt that a subject can wear around the waist or the like, an air bag (pressure sensitive portion) that is attached to a surface of the belt, and a pressure sensor that detects the internal pressure of the air bag. In a state in which a subject wears the belt, when a body motion of the subject acts on the air bag, the internal pressure of the air bag changes. Based on the internal pressure, the pressure sensor acquires signals related to biological information of the subject (for example, a body motion such as rolling over in bed, a breathing motion, a heartbeat, and the like of the subject).

### CITATION LIST

### Patent Literature

PTL 1: Japanese Unexamined Patent Application Publication No. 2001-286448

### SUMMARY OF INVENTION

### Technical Problem

In order that the biological information acquisition device PTL 1 can acquire biological information of a subject, the subject has to additionally wear the belt. Therefore, the subject tends to feel a sensation of tightness due to wearing of the belt.

The present invention has been made against such a background, and an object of the present invention is to provide a biological information acquisition device that can reliably acquire biological information of a subject while suppressing a sensation of tightness experienced by the subject.

### Solution to Problem

A first invention is a biological information acquisition device including: a body motion detector (40) that includes a pressure sensitive portion (41, 91) on which a body motion of a subject acts and that detects a signal indicating the body motion of the subject; and a clamp member (11) that clamps a clothing item (2, 3) of the subject. The body motion detector (40) includes a hollow member (41, 91) that is the pressure sensitive portion, and a pressure sensor (42) that detects a pressure of the hollow member (41, 91) as the signal. The clamp member (11) includes a first member (20, 90) that is disposed on the subject side of the clothing item (2, 3), and a second member (30) that is disposed on an opposite side of the clothing item (2, 3) from the first member (20, 90). The pressure sensitive portion (41, 91) is disposed on the first member (20, 90) side.

With the first invention, the biological information acquisition device (10) is attached to the clothing item (2, 3) (such as a belt, a skirt, trousers, a shoe, or disposable underpants) of the subject by clamping the clothing item (2, 3) by using the clamp member (11). That is, with the present invention, it is not necessary for the subject to additionally wear a belt or the like in order to acquire biological information as in existing technologies, and the biological information acquisition device (10) may be attached to the clothing item (2, 3), which is worn by the subject, via the clamp member (11). Accordingly, the subject does not experience a strong sensation of tightness.

In the clamp member (11), the pressure sensitive portion (41, 91) is disposed in the first member (20) that is positioned on the subject side. Therefore, a body motion of the subject can be reliably transmitted to the pressure sensitive portion (41, 91). The body motion detector (40) detects a signal in accordance with the body motion that acts on the pressure sensitive portion (41, 91). As a result, biological information of the subject can be acquired based on the signal.

With the first invention, the body motion detector (40), which detects the signal indicating a body motion of the subject, includes the hollow member (41, 91), which is the pressure sensitive portion, and the pressure sensor (42). Because the hollow member (41, 91) is disposed in the first member (20), the body motion of the subject can easily act on the hollow member (41, 91). The pressure sensor (42) detects a signal indicating the body motion based on the internal pressure of the hollow member (41, 91).

A second invention includes a circuit board (50) that performs signal processing for acquiring biological information of the subject based on the signal detected by the body motion detector (40), and the circuit board (50) is not disposed on the first member (20, 90) side.

With the second invention, because the circuit board (50) is not disposed in the first member (20) on the subject side, the first member (20) can be reduced in size and thickness. Thus, it is possible to suppress a sensation of tightness or an uncomfortable sensation that is experienced by the subject due to the presence of the first member (20).

A third invention is the biological information acquisition device according to the second invention, in which the circuit board (50) is disposed on the second member (30) side.

With the third invention, the circuit board (50) is disposed in the second member (30) of the clamp member (11), which is separated from the subject. Therefore, it is possible to suppress a sensation of tightness or an uncomfortable sensation experienced by the subject due to the presence of the circuit board (50).

A fourth invention is the biological information acquisition device according to any one of the first to third inventions, including an elastic member (25, 85) that urges the clamp member (11) in such a way that the first member (20, 90) and the second member (30) become closer to each other.

With the fourth invention, in a state in which the clothing item (2, 3) is clamped by the clamp member (11), the elastic member (25, 85) urges the first member (20, 90) and the second member (30) in such a way that the first member (20, 90) and the second member (30) become closer to each other. Therefore, unintended removal of the clamp member (11) from the clothing item (2, 3) can be prevented.

A fifth invention is the biological information acquisition device according to the fourth invention, in which the clamp member (11) includes a connection member (15) that couples an end portion of the first member (20) and an end portion of the second member (30) to each other, and in which the elastic member (25, 85) is a plate spring that extends along the first member (20, 90), the connection member (15), and the second member (30) and that has a U-shaped thickness cross section.

With the fifth invention, the U-shaped plate spring (25), which is an elastic member, is disposed so as to extend along the first member (20, 90), the second member (30), and the connection member (15) of the clamp member (11). In the clamp member (11), due to elasticity of the plate spring (25), the first member (20, 90) and the second member (30) are urged in a direction such that the first member (20, 90) and the second member (30) become closer to each other with the connection member (15) as a fulcrum. Therefore, the clamp member (11) can strongly clamp the clothing item (2, 3), and unintended removal of the clamp member (11) from the clothing item can be prevented.

A sixth invention is the biological information acquisition device according to any one of the first to fifth inventions, in which the first member (20) is a casing (20) that contains the hollow member (41).

With the sixth invention, the hollow member (41, 91) is contained in the casing (20), which is the first member. Therefore, breakage or damage of the hollow member (41, 91) can be prevented. Thus, it is possible to avoid reduction in accuracy of biological information due to breakage or damage of the hollow member (41, 91).

A seventh invention is the biological information acquisition device according to the sixth invention, in which the casing (20) includes a first wall (61) on which a body motion of the subject acts, and a second wall (62) that is disposed on an opposite side of the hollow member (41) from the first wall (61), and in which a rigidity of the first wall (61) is lower than a rigidity of the second wall (62).

With the seventh invention, the rigidity of the first wall (61) of the casing (20), which is on the subject side of the hollow member (41, 91), is low. Therefore, for example, when a body motion of the subject acts on the first wall (61), the first wall (61) deforms, and due to the deformation, the internal pressure of the hollow member (41, 91) easily changes. As a result, the accuracy of biological information is improved.

Moreover, with the present invention, the rigidity of the second wall (62) of the casing (20), which is on the opposite side of the hollow member (41, 91) from the subject, is high. Therefore, even if a body motion of the subject acts on the second wall (62) via the hollow member (41, 91), the second wall (62) does not considerably deform. If the second wall (62) deforms, the internal pressure of the hollow member (41, 91) does not change easily, and the accuracy of biological information may decrease. By preventing deformation of the second wall (62), the accuracy of biological information is further improved.

An eighth invention is the biological information acquisition device according to the sixth invention, in which the casing (20) includes a first wall (61) on which a body motion of the subject acts, a second wall (62) that is disposed on an opposite side of the hollow member (41) from the first wall (61), and in which a peripheral wall (74) that is disposed between the first wall (61) and the second wall (62) and that includes a small-thickness portion (78) having a thickness smaller than a thickness of each of the first wall (61) and the second wall (62).

With the eighth invention, the small-thickness portion (78) is formed in the peripheral wall (74) of the casing (20) between the first wall (61) and the second wall (62). Therefore, when a body motion of the subject acts on the first wall (61), the peripheral wall (74) can easily deform as deformation starts from the small-thickness portion (78). Thus, the first wall (61) can more easily deform in the thickness direction thereof, and the pressing force that the first wall (61) applies to the hollow member (41, 91) increases. As a result, the internal pressure of the hollow member (41, 91) can easily change due to the body motion of the subject, and the accuracy of biological information is improved.

A ninth invention is the biological information acquisition device according to the sixth invention, in which the casing (20) includes a wall (61) on which a body motion of the subject acts, and a support portion (65) that supports the wall (61) in such a way that the wall (61) is displaceable toward the hollow member (41).

With the ninth invention, when a body motion of the subject acts on the wall (61), the wall (61) becomes displaced toward the hollow member (41, 91). As a result, the internal pressure of the hollow member (41, 91) can easily change due to the body motion of the subject, and the accuracy of biological information is improved.

A tenth invention is the biological information acquisition device according to any one of the first to fifth inventions, in which at least a part of the first member (20) also serves as the hollow member (91).

With the tenth invention, at least a part of the first member (20) is the hollow member (91) having a hollow shape. Because the hollow member (91) is disposed on the subject side, a body motion of the subject can be reliably made to act on the hollow member (91).

Moreover, the hollow member (91) is used as both of the pressure sensitive portion on which a body motion of the subject acts and the clamp member (11) for clamping the clothing item (2, 3). Thus, the number of components can be reduced, and the biological information acquisition device can be simplified.

### Advantageous Effects of Invention

With the present invention, the biological information acquisition device (10) is worn by a subject by clamping the clothing item (2, 3) of the subject with the clamp member (11). Therefore, it is not necessary for the subject to wear an additional clothing item as in existing technologies, and a sensation of tightness experienced by the subject can be reduced.

If the subject experiences a sensation of tightness when acquiring biological information, this may affect the accuracy of biological information. To be specific, for example, when acquiring autonomic nerve information (such as an indicator of stress) as biological information, if the subject wears an additional belt, this may affect the autonomic nerve information. However, with the present invention, because a sensation of tightness experienced by the subject can be suppressed, decrease of the accuracy of biological information due to a sensation of tightness can be also prevented.

A subject can wear the biological information acquisition device (10) by only clamping the clothing item (2, 3) with the clamp member (11). Therefore, it is easy to wear and remove the biological information acquisition device (10).

In the clamp member (11), the pressure sensitive portion (41, 91) is disposed on the first member (20) side, which is near a subject. Therefore, a body motion of the subject can be made to reliably act on the pressure sensitive portion (41, 91), and the accuracy of detection of biological information can be further improved.

### BRIEF DESCRIPTION OF DRAWINGS

FIG. 1 is an external top view of a biological information acquisition device according to a first embodiment.

FIG. 2 is an external front view of the biological information acquisition device according to the first embodiment.

FIG. 3 is an external right side view of the biological information acquisition device according to the first embodiment.

FIG. 4 is a sectional view taken along line IV-IV of FIG. 1.

FIG. 5 is a sectional view taken along line V-V of FIG. 3.

FIG. 6 is a block diagram illustrating the overall structure of the biological information device according to the first embodiment.

FIG. 7 is an external right side view of a biological information acquisition device according to a modification of the first embodiment.

FIG. 8 is a cross-sectional view of a first member of the biological information acquisition device according to the modification of the first embodiment, illustrating a state in which a body motion of a subject is not acting on a tube.

FIG. 9 is a cross-sectional view of the first member of the biological information acquisition device according to the modification of the first embodiment, illustrating a state in which a body motion of a subject is acting on the tube.

FIG. 10 is an external right side view of a biological information acquisition device according to a second embodiment.

FIG. 11 is a longitudinal sectional view (corresponding to FIG. 4) of the biological information acquisition device according to the second embodiment.

FIG. 12 is a cross-sectional view of a first member of the biological information acquisition device according to the second embodiment, illustrating a state in which a body motion of a subject is not acting on a tube.

FIG. 13 is a cross-sectional view of the first member of the biological information acquisition device according to the second embodiment, illustrating a state in which a body motion of a subject is acting on the tube.

FIG. 14 is a front view of a biological information acquisition device according to a third embodiment.

FIG. 15 is a longitudinal sectional view (corresponding to FIG. 4) of the biological information acquisition device according to the third embodiment.

FIG. 16 is a cross-sectional view of a first member of the biological information acquisition device according to the third embodiment, illustrating a state in which a body motion of a subject is not acting on a tube.

FIG. 17 is a cross-sectional view of the first member of the biological information acquisition device according to the third embodiment, illustrating a state in which a body motion of a subject is acting on the tube.

FIG. 18 is a side view illustrating a schematic structure of a biological information acquisition device according to a first example, which is one of other examples.

FIG. 19 is a side view illustrating a schematic structure of a biological information acquisition device according to a second example, which is one of other examples.

FIG. 20 is a side view illustrating a schematic structure of a biological information acquisition device or a unit according to a third example, which is one of other examples.

FIG. 21 is a schematic side view illustrating a schematic structure of a biological information acquisition device or a unit according to a fourth example, which is one of other examples.

### DESCRIPTION OF EMBODIMENTS

Hereafter, embodiments of the present invention will be described in detail with reference to the drawings. The embodiments described below are essentially preferred examples, and the embodiments are not intended to limit the present invention, applications of the present invention, and the range of use of the present invention.

### First Embodiment of Invention

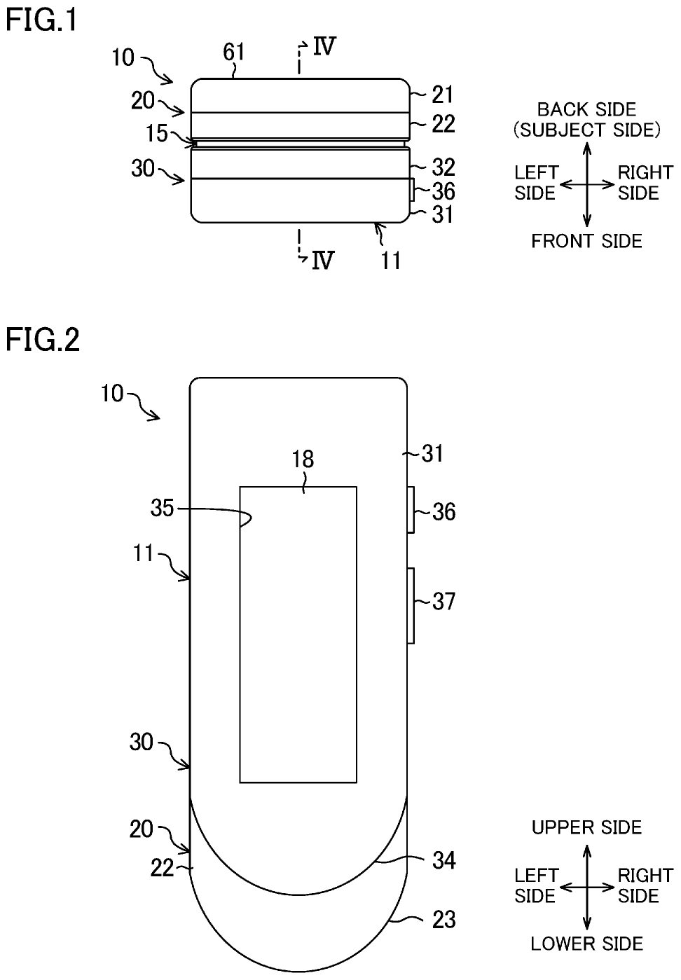

A biological information acquisition device according to a first embodiment (see FIGS. 1 to 6) is a biological information acquisition device (10) that is wearable by a subject. The biological information acquisition device (10) is attachable to or removable from a clothing item (2, 3) of a subject, and is attached to the subject via the clothing item (2, 3). The biological information acquisition device (10) according to the present embodiment is attached to a belt (2) and trousers (3), which are clothing items.

The biological information acquisition device (10) includes a case unit (11), a pressure sensing unit (40), a circuit board (50), and a rechargeable battery (45).

### Overall Structure of Case Unit

As illustrated in FIG. 1, the external shape of the case unit (11) is an elongated inverted U-shape whose lower side is open. The case unit (11) corresponds to a clamp member that clamps the clothing item (2, 3) of the subject. The case unit (11) includes a first casing (20), a second casing (30), and a connection member (15). The first casing (20) corresponds to a first member that is disposed on the subject side of the clothing item (2, 3). The second casing (30) corresponds to a second member that is disposed on the opposite side of the clothing item (2, 3) from the subject. The connection member (15) couples an end portion of the first casing (20) and an end portion of the second casing (30) to each other.

The case unit (11) includes a recessed portion (12) (insertion hole), through which the clothing item (2, 3) is inserted, among the first casing (20), the second casing (30), and the connection member (15). A clip (25), which is a plate spring, is contained in the case unit (11).

### First Casing

The external shape of the first casing (20) is a substantially rectangular shape that is elongated in the up-down direction. In the present embodiment, the height of the entirety of the first casing (20) in the up-down direction is larger than the height of the entirety of the second casing (30) in the up-down direction. The thickness of the first casing (20) in the front-back direction is substantially equal to or larger than the thickness of the second casing (30) in the front-back direction. The first casing (20) includes a back case (21) in a back part (rear part) thereof. The first casing (20) includes a first middle case (22) in a front part (face part) thereof. The first casing (20), which has a hollow shape, is formed by integrally fixing the back case (21) and the first middle case (22) to each other. A first space (S1) is formed in the first casing (20).

The back case (21) in the present embodiment is made of a comparatively flexible material (such as an elastomer resin). In contrast, the first middle case (22) is made of an ABS resin.

The first casing (20) includes, at a lower end portion thereof, a first budging portion (23) that bulges downward in an arc shape.

The first casing (20) includes, at an upper end portion thereof, a first protrusion (24) that protrudes forward. The first protrusion (24) has a rectangular tubular shape that protrudes forward from an upper end front portion of the first middle case (22). One end (back end) of the connection member (15) is fixed to the first protrusion (24).

### Second Casing

The external shape of the second casing (30) is a substantially rectangular shape that is elongated in the up-down direction. The second casing (30) includes a front case (31) in a front part thereof. The second casing (30) includes a second middle case (32) in a back part thereof. The second casing (30), which has a hollow shape, is formed by integrally fixing the front case (31) and the second middle case (32) to each other. A second space (S2) is formed in the second casing (30).

The front case (31) and the second middle case (32) are made of, for example, an ABS resin.

The second casing (30) includes, in a lower end portion thereof, a second budging portion (34) that bulges downward in an arc shape.

In the front surface of the second casing (30), an opening (35) for exposing a liquid crystal display (18), which is a display portion, is formed. The liquid crystal display (18) displays information related to the operation of the biological information acquisition device (10) and information related to biological information of a subject. A body portion of the liquid crystal display (18) is contained in the second space (S2).

A power button (36) and a switching button (37) are disposed on a right side surface of the second casing (30). The power button (36) is a switch for switching on and off the liquid crystal display (18) or the biological information acquisition device (10). The switching button (37) is an operation portion for switching, for example, between items displayed on the liquid crystal display (18).

The second casing (30) includes, at an upper end portion thereof, a second protrusion (38) that protrudes backward. The second protrusion (38) has a rectangular tubular shape that protrudes backward from an upper end back portion of the first middle case (22). The other end (front end) of the connection member (15) is fixed to the second protrusion (38).

### Connection Member

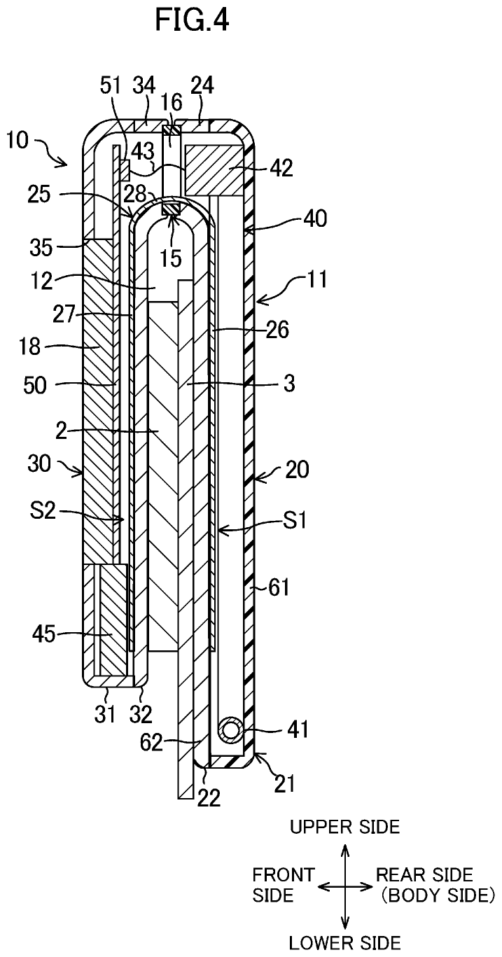

As illustrated in FIG. 4, the connection member (15) is disposed between the first protrusion (24) of the first casing (20) and the second protrusion (38) of the second casing (30). The connection member (15) has a rectangular tubular shape having an axis extending in the front-back direction and is made of a flexible resin material. Thus, the first casing (20) and the second casing (30) can tilt with the connection member (15) as a fulcrum. One end (back end) of the connection member (15) in the axial direction is fitted into the first protrusion (24). The other end (front end) of the connection member (15) in the axial direction is fitted into the second protrusion (38). An inner space (16) having a rectangular shape is formed in the connection member (15).

### Clip

The clip (25) is contained in the case unit (11). The clip (25) corresponds to an elastic member that urges the case unit (11) in such a way that the first casing (20) and the second casing (30) become closer to each other. The clip (25) is a plate spring that extends along the first casing (20), the connection member (15), and the second casing (30) and that has a U-shaped thickness cross section.

The clip (25) includes a first flat plate (26) that is disposed in the first space (S1) of the first casing (20), a second flat plate (27) that is disposed in the second space (S2) of the second casing (30), and an arc-shaped plate (28) that connects an upper end of the first flat plate (26) and an upper end of the second flat plate (27). The first flat plate (26) is in surface-contact with an inner surface of the first middle case (22) in an upright state. The second flat plate (27) is in surface-contact with an inner surface of the second middle case (32) in an upright state. The arc-shaped plate (28) has an arc-shaped surface shape that bulges upward so as to be in contact with a lower edge portion of each of the first protrusion (24), the connection member (15), and the second protrusion (38).

The clip (25) urges the first middle case (22) and the second middle case (32) so as to hold these cases from both sides. To be specific, the first flat plate (26) urges the first middle case (22) forward, and the second flat plate (27) urges the second middle case (32) backward. Thus, the first casing (20) and the second casing (30) tilt so as to become closer to each other with the connection member (15) as a fulcrum. As a result, the clothing item (2, 3), which is inserted through the recessed portion (12) of the case unit (11), is clamped between the first casing (20) and the second casing (30).

### Pressure Sensing Unit

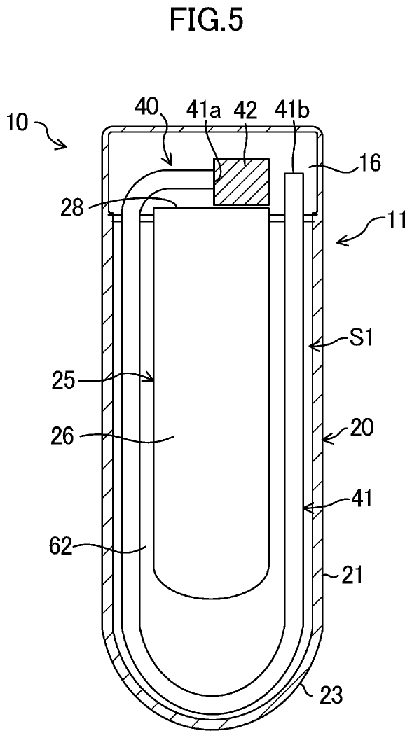

As illustrated in FIGS. 4 and 5, the pressure sensing unit (40) is contained in the first space (S1) of the first casing (20). The pressure sensing unit (40) corresponds to a body motion detector that detects a signal indicating a body motion of a subject. The pressure sensing unit (40) in the present embodiment includes a tube (41) and a pressure sensor (42).

The tube (41) corresponds to a pressure sensitive portion on which a body motion of a subject acts, and is a hollow member having a hollow shape. To be specific, the tube (41) has an elongated tubular shape having a circular cross section, and is made of a flexible resin material (such as vinyl chloride). The tube (41) is disposed in the back case (21) of the first casing (20).

To be specific, the tube (41) is disposed in a substantially U-shape along an inner peripheral wall of the back case (21). A proximal end portion (41a) and a distal end portion (41b) of the tube (41) are positioned near an upper end of the first casing (20). The proximal end portion (41a) of the tube (41) is connected to the pressure sensor (42) via a tube connector (not shown). The distal end portion (41b) of the tube (41) may be open in the first space (S1), or the distal end portion (41b) may be closed with a plug. The plug may have a communication hole through which the inside and the outside of the tube (41) communicate with each other. In either case, the tube (41) is structured in such a way that the internal pressure thereof changes when a body motion of a subject acts on the tube (41).

The pressure sensor (42) is disposed at a position that is near the upper end of the first casing (20) and that overlaps the connection member (15) in the front-back direction. The pressure sensor (42) includes a microphone. The pressure sensor (42) detects the internal pressure of the tube (41), and outputs a pressure signal in accordance with the internal pressure. The pressure signal is a signal indicating a body motion of a subject.

As illustrated in FIG. 4, a signal wire (43), for transmitting a detected pressure signal to the circuit board (50), is connected to the pressure sensor (42). The signal wire (43) extends toward the circuit board (50) via the inner space (16) of the connection member (15).

### Circuit Board

As illustrated in FIG. 4, the circuit board (50) is contained in the second space (S2) of the second casing (30). The circuit board (50) is a printed circuit board on which an electronic circuit that includes a central processing unit (CPU) and storage devices (such as a memory and a register) are mounted. The circuit board (50) has a plate-like shape that extends in the up-down direction along the front surface and the back surface of the second casing (30).

The liquid crystal display (18) described above is mounted on the front surface (surface) of the circuit board (50). A signal wire connection terminal (51), to which the signal wire (43) is connected, is disposed on an upper part of the back surface of the circuit board (50).

### Rechargeable Battery

As illustrated in FIG. 4, the rechargeable battery (45) is contained in the second space (S2) of the second casing (30). The rechargeable battery (45) is an electric power source of the biological information acquisition device (10). The rechargeable battery (45) is charged, as necessary, by receiving electricity from an electric power supply device (not shown).

### Functional Blocks

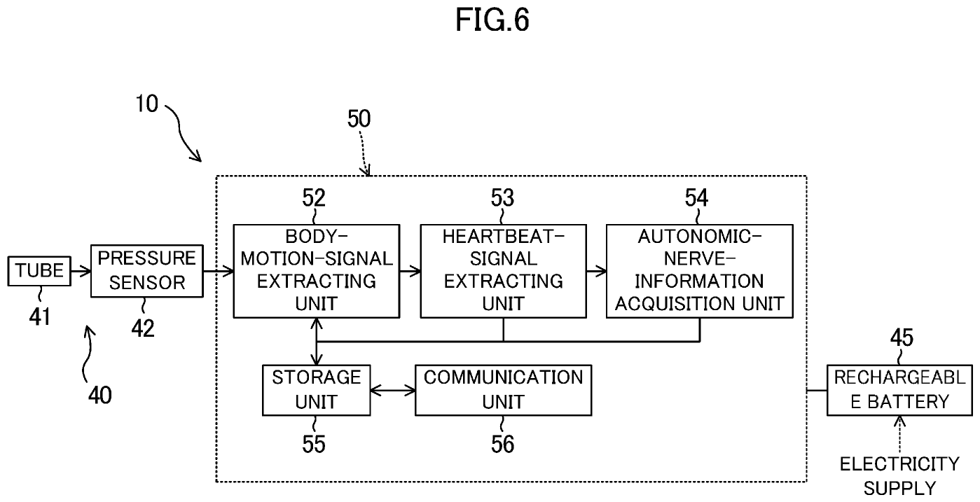

Referring to FIG. 6, functional blocks included in the circuit board (50) will be described in detail. The biological information acquisition device (10) includes a body-motion-signal extracting unit (52), a heartbeat-signal extracting unit (53), an autonomic-nerve-information acquiring unit (54), a storage unit (55), and a communication unit (56).

The body-motion-signal extracting unit (52) extracts a body motion signal of a subject based on a signal output from the pressure sensor (42). Here, a body motion signal is a signal in which a large body motion (rough body motion) of a subject and a small body motion (slight body motion) due to heartbeat and breathing of the subject are superposed. The heartbeat-signal extracting unit (53) extracts only a signal due to heartbeat (heartbeat signal) from a body motion signal extracted by the body-motion-signal extracting unit (52).

The autonomic-nerve-information acquiring unit (54) calculates, for example, the LF/HF value of a subject based on a heartbeat signal extracted by the heartbeat-signal extracting unit (53). Here, the LF/HF value is the ratio of a low-frequency component LF of heartbeat-interval fluctuation, which is obtained based on the heartbeat signal, to a high-frequency component HF of the heartbeat interval fluctuation. The LF/HF value is an indicator of stress level and autonomic nerve activity. A higher LF/HF value indicates a higher stress level.

The storage unit (55) includes a semiconductor memory such as a flash memory. The storage unit (55) stores signals that are processed in the circuit board (50) and data related to acquired biological information together with the corresponding time as necessary. To be specific, for example, the storage unit (55) successively stores the body motion signal, the heartbeat signal, the LF/HF value, and the like as time-series data.

The communication unit (56) is a communication interface of the biological information acquisition device (10). That is, the communication unit (56) is connected to an external device (an information terminal such as a smartphone, a tablet, or a personal computer) via wired or wireless network communication. For example, each of information items stored in the storage unit (55) is transmitted to the external device via network communication. Thus, a subject can check details of biological information by using the external device.

### Attachment/Removal of Biological Information Acquisition Device

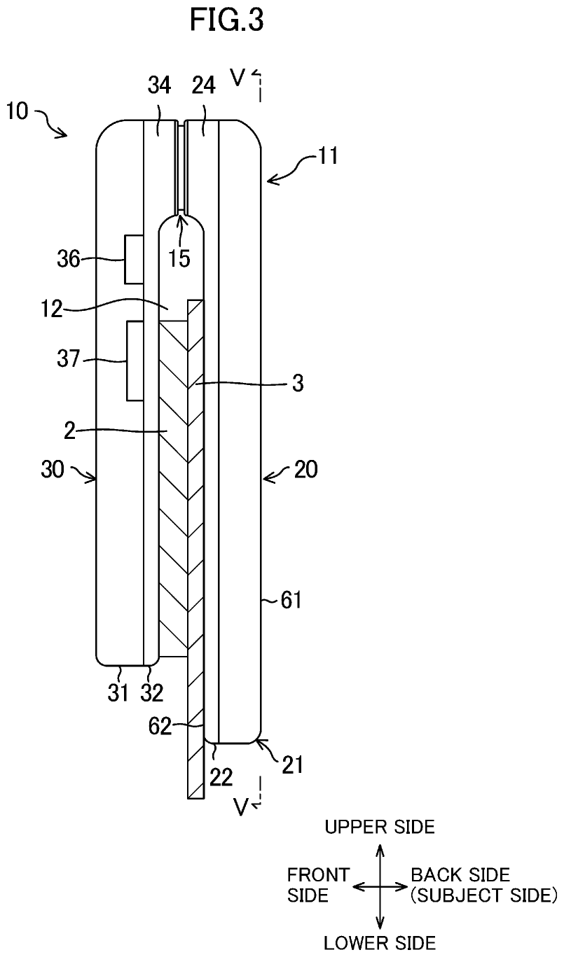

As illustrated in FIGS. 3 and 4, the biological information acquisition device (10) according to the present embodiment is worn by a subject by attaching the case unit (11) to the clothing item (2, 3). To be specific, the belt (2) and a part of the trousers (3) around the waist are simultaneously inserted through the recessed portion (12) of the case unit (11). In the state in which the belt (2) and the trousers (3) are inserted through the recessed portion (12), the first casing (20) and the second casing (30) are urged by the clip (25) that is disposed therein. Thus, the belt (2) and the trousers (3) are clamped by the first casing (20) and the second casing (30). Accordingly, unintended removal of the case unit (11) from the belt (2) and the trousers (3) can be reliably prevented.

When removing the biological information acquisition device (10), the first casing (20) and the second casing (30) are separated from each other against the urging force of the clip (25), and the case unit (11) is moved upward. Thus, the biological information acquisition device (10) can be easily removed from the belt (2) and the trousers (3).

### Operation of Biological Information Acquisition Device

In a state in which the biological information acquisition device (10) is attached to the belt (2) and the trousers (3), the abdomen of a subject is in contact with the first casing (20). Therefore, a body motion of the subject is transmitted to the tube (41) via a first wall (61) of the first casing (20). The pressure sensor (42) detects the internal pressure of the tube (41) or change in the internal pressure, and outputs a pressure signal. In the circuit board (50), a body motion signal and a heartbeat signal are extracted in this order from the pressure signal, and the LF/HF value of the subject is successively calculated.

### Advantageous Effects of First Embodiment

Because the biological information acquisition device (10) according to the present embodiment is attached to the belt (2), the trousers (3), and the like, which are worn by a subject, it is not necessary for the subject to additionally wear a proprietary belt as in existing technologies. Therefore, when the subject wears the biological information acquisition device (10), a sensation of tightness or an uncomfortable sensation experienced by the subject can be suppressed.

As described above, a subject can easily wear the biological information acquisition device (10) by inserting the belt (2) and the trousers (3) through the recessed portion (12) of the case unit (11). Because the case unit (11) strongly clamps the belt (2) and the trousers (3) with the clip (25), unconscious removal of the biological information acquisition device (10) from the belt (2) and the trousers (3) can also be prevented. Accordingly, biological information of the subject can be reliably acquired.

The tube (41), to which a body motion of a subject is transmitted, is disposed in the first casing (20), which is a part of the case unit (11) that is positioned on the subject side. Therefore, the body motion of the subject can be easily transmitted to the tube (41), and the accuracy of the LF/HF value, which is biological information, is improved.

Because the tube (41) is contained in the first casing (20), the tube (41) can be protected by the first casing (20). Accordingly, damage and breakage of the tube (41) can be prevented, and the reliability of the biological information acquisition device (10) can be improved.

On the other hand, the circuit board (50) for performing signal processing is disposed in the second casing (30), which is separated from a subject. That is, the circuit board (50) is not disposed in the first casing (20) on the subject side. Therefore, the first casing (20), which is positioned on the subject side, can be reduced in size and thickness, and a sensation of tightness experienced by the subject can be further suppressed.

In the first casing (20), the rigidity of the first wall (61), which has a pressure-receiving surface, is lower than the rigidity of a second wall (62), which has a support surface. Therefore, when a body motion of a subject acts on the first wall (61), the first wall (61) easily deforms due to the body motion, and the degree of deformation of the tube (41), which is pressed against the first wall (61), increases. Thus, the internal pressure of the tube (41) easily changes in accordance with the body motion of the subject, and therefore the pressure signal, that is, the degree of change in body motion signal becomes larger. Accordingly, the accuracy of biological information acquired by the biological information acquisition device (10) is improved.

By increasing the rigidity of the second wall (62), it is possible to suppress deformation of the second wall (62), which supports the tube (41), due to a body motion of a subject acting on the tube (41). Thus, the tube (41) can be deformed to a sufficient degree, and the accuracy of biological information is improved.

### Modification of First Embodiment

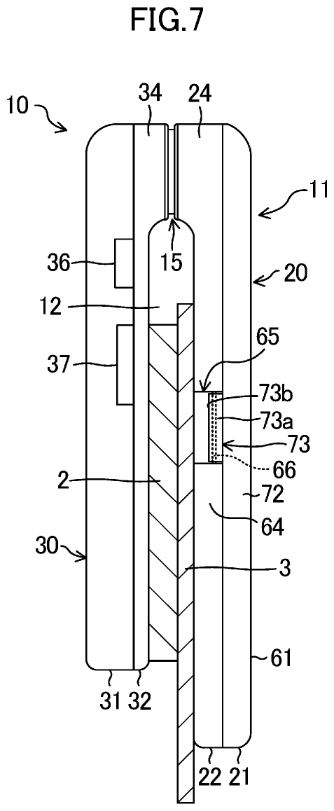

FIGS. 7 to 9 illustrate a modification of the first embodiment described above, which differs from the first embodiment in the structure of the first casing (20). In the first casing (20) of the present modification, the back case (21) and the first middle case (22) are made of the same material (such as an ABS resin).

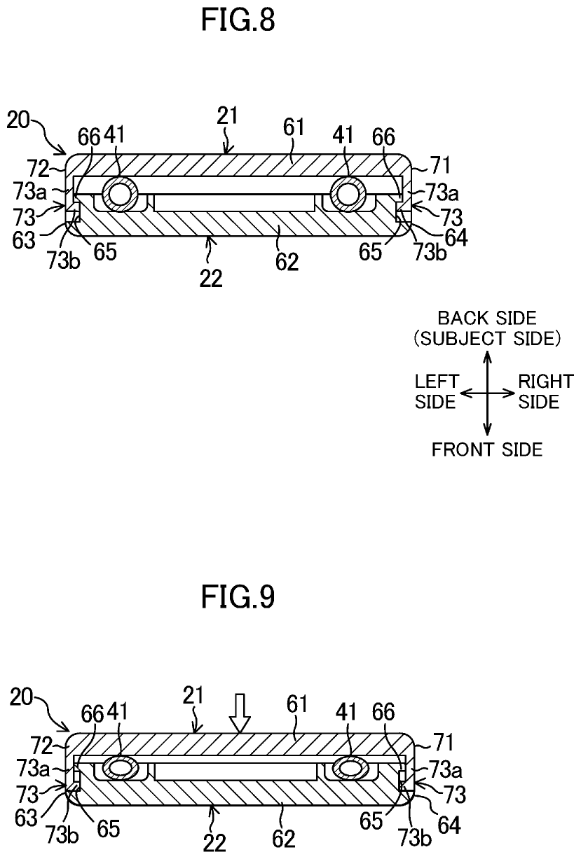

At least left and right side walls (63, 64) of the first middle case (22) include engagement grooves (65) having rectangular shapes. The first middle case (22) includes groove-side protrusions (66) that are on the rear side of the engagement grooves (65) and that protrude outward in the left-right direction (see FIG. 8). The engagement grooves (65) and the groove-side protrusions (66) may be included in the upper wall and the lower wall of the first middle case (22).

At least left and right side walls (71, 72) of the back case (21) include engagement portions (73) that correspond to the engagement grooves (65). The engagement portions (73) include protruding pieces (73a), which protrude forward from the body of the back case (21), and claw portions (73b), which have hook-like shapes that are curved inward in the left-right direction from the distal ends of the protruding pieces (73a). The engagement portions (73) are engaged with the engagement grooves (65) in such a way that each of the claw portions (73b) extends over a corresponding one of the groove-side protrusions (66). The thickness of each of the claw portions (73b) in the front-back direction is smaller than the length of each of the engagement grooves (65) in the front-back direction. Therefore, the claw portions (73b) of the engagement portions (73) are displaceable in the engagement grooves (65) in the front-back direction.

Because the engagement portions (73) are engaged with the engagement grooves (65) in this way, a wall (the first wall (61) of the back case (21)) on which a body motion of a subject acts is supported by the engagement grooves (65), which are support portions, in such a way that the wall is displaceable toward the tube (41).

In the present modification, when a body motion of a subject acts in the direction of an arrow in FIG. 9, the engagement portions (73) of the back case (21) become displaced forward in the engagement grooves (65). Thus, the tube (41) is pressed by the back case (21), the tube (41) deforms, and the internal pressure of the tube (41) changes. Thus, with the present modification, the tube (41) can be deformed to a sufficient degree without reducing the rigidity of the back case (21). The other advantageous effects are the same as those of first embodiment.

### Second Embodiment of Invention

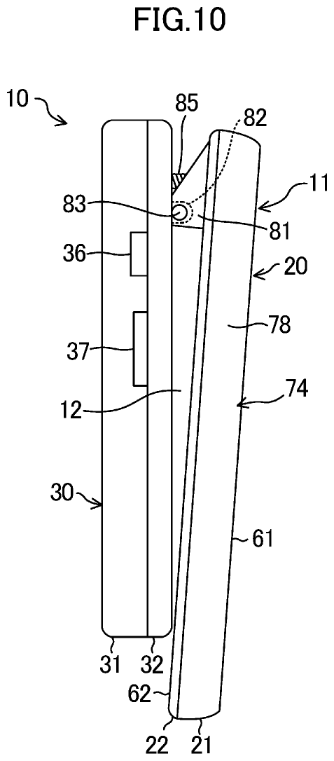

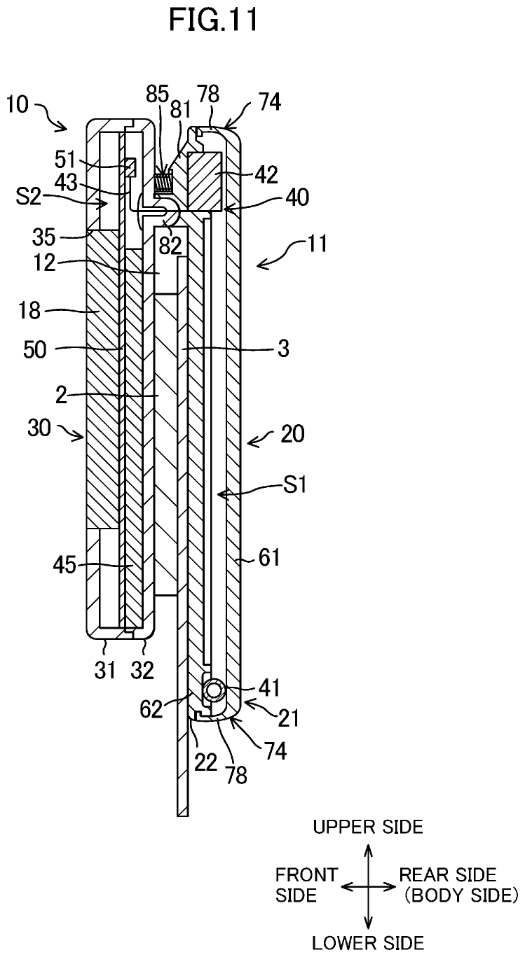

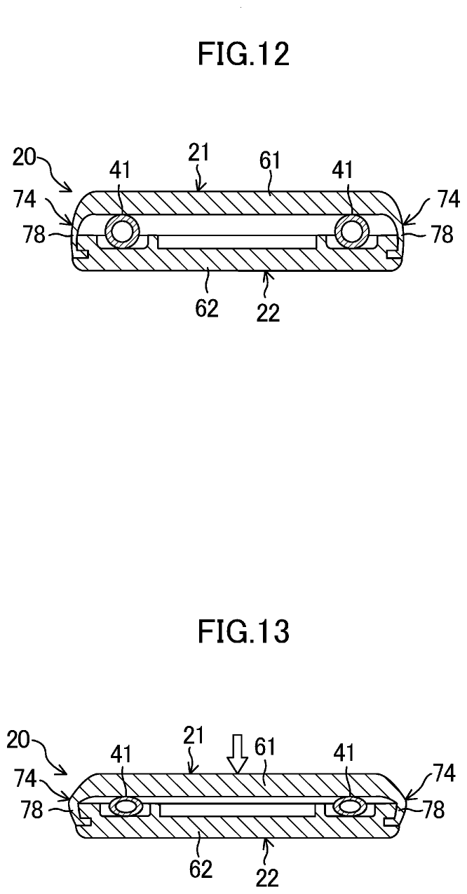

FIGS. 10 to 13 illustrate a second embodiment that differs from the first embodiment in the structure of the case unit (11). The case unit (11) of the second embodiment includes a bracket (81), instead of the first protrusion (24) of the first casing (20). The bracket (81) has a substantially triangular-prismatic shape that protrudes forward from an upper end portion of the first middle case (22).

The second casing (30) includes a pivot support (82) instead of the second protrusion (38). The pivot support (82) protrudes forward from an upper end portion of the second middle case (32) so as to overlap the bracket (81) of the first casing (20) in the left-right direction. The pivot support (82) and the bracket (81) are coupled to each other via a pin (83). Thus, the first casing (20) and the second casing (30) can rotate relative to each other with the pin (83) as a fulcrum.

The clip (25) of the first embodiment is not disposed in the case unit (11) of the second embodiment. In the second embodiment, a spring (85) is used as an elastic member, instead of the clip (25). The spring (85) is interposed, outside of the case unit (11), between an upper end portion of the first casing (20) and an upper end portion of the second casing (30). To be more specific, the spring (85) is disposed slightly above the bracket (81) and the pivot support (82), one end of the spring (85) is coupled to the first middle case (22), and the other end of the spring (85) is coupled to the second middle case (32). The spring (85) extends in a direction such that the upper end portion of the first casing (20) and the upper end portion of the second casing (30) become separated from each other. As a result, a body portion of the first casing (20) and a body portion of the second casing (30) become closer to each other. That is, the spring (85) is an elastic member that urges the first casing (20) and the second casing (30) in a direction such that the first casing (20) and the second casing (30) become closer to each other.

The rechargeable battery (45) of the second embodiment is longer than that of the first embodiment in the up-down direction, and is disposed on the back side of the circuit board (50).

In the first casing (20) of the second embodiment, the back case (21) and the first middle case (22) are made of the same material (such as an ABS resin). The back case (21) of the second embodiment includes a small-thickness portion (78) around all of a peripheral wall (74) of the first wall (61) (a peripheral wall constituted by an upper wall, a lower wall, a right wall, and a left wall of the back case (21)). The small-thickness portion (78) is a fragile portion whose thickness is smaller than that of the first wall (61).

### Advantageous Effects of Second Embodiment

With the second embodiment, the spring (85) urges the first casing (20) and the second casing (30) in a direction such that the first casing (20) and the second casing (30) become closer to each other with the pin (83) as a fulcrum. Therefore, the clothing item (2, 3) can be strongly held between the first casing (20) and the second casing (30). Accordingly, unintended removal of the biological information acquisition device (10) from the clothing item (2, 3) can be reliably prevented.

With the second embodiment, when a body motion of a subject acts in the direction of an arrow in FIG. 13, the small-thickness portion (78) of the first casing (20) flexurally deforms, and the first wall (61) deforms toward the tube (41). Thus, the tube (41) is pressed by the back case (21), the tube (41) deforms, and the internal pressure of the tube (41) changes. Thus, also with the second embodiment, the tube (41) can be deformed to a sufficient degree. The other advantageous effects are the same as those of first embodiment.

### Third Embodiment of Invention

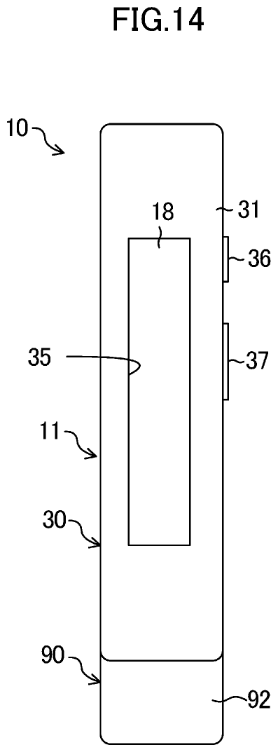

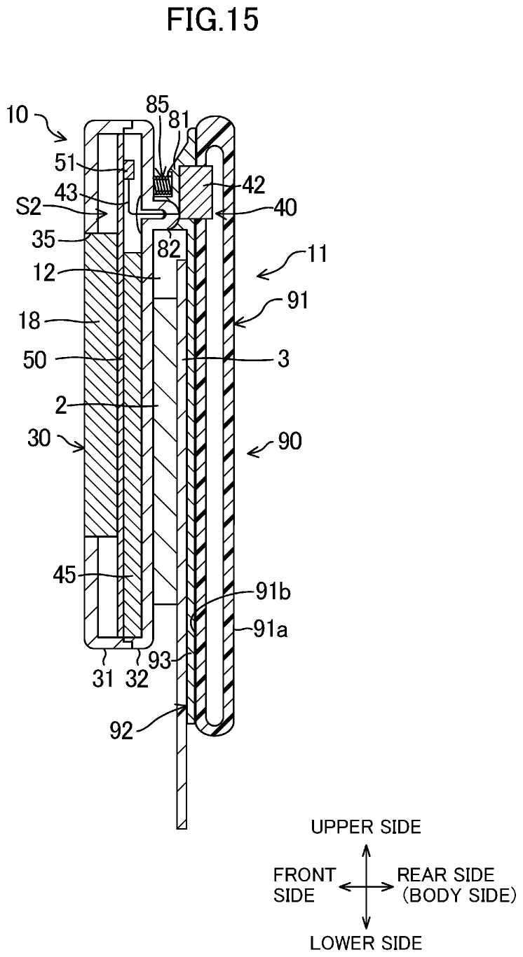

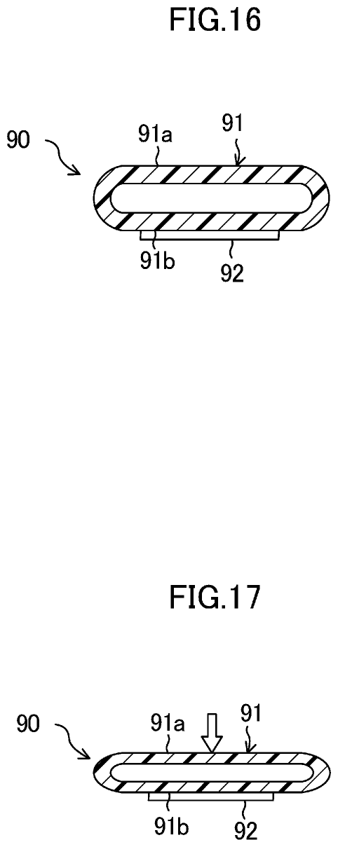

FIGS. 14 to 17 illustrate a third embodiment that differs from the first and second embodiments in the structure of the case unit (11). The width of the case unit (11) of the third embodiment in the left-right direction is small (see FIG. 14), compared with the first and second embodiments.

In the third embodiment, a tube unit (90) corresponds to a first member of the case unit (11), which is positioned on the subject side. The tube unit (90) includes a flat tube (91), which is disposed on the subject side, and a middle support portion (92), which is disposed on a side opposite from the subject.

The flat tube (91) is a pressure sensitive portion on which a body motion of a subject acts, and is a hollow member having a hollow shape. The flat tube (91) is used as a part of the first member. The width of the flat tube (91) is larger than that of the tube (41) of the first and second embodiments. The cross section of the flat tube (91) has an elongated circular shape or an elliptical shape that is elongated in the left-right direction. Both ends of the flat tube (91) in the axial direction are closed. A back-surface portion (91a) of the flat tube (91) is a pressure-receiving surface on which a body motion of a subject acts. A front-surface portion (91b) of the flat tube (91) is in contact with the middle support portion (92). A holding hole (91c), into which the pressure sensor (42) is fitted, is formed in an upper part of a surface of the flat tube (91). With this structure, the pressure sensor (42) can detect the internal pressure of the flat tube (91).

The middle support portion (92) includes a support plate (93), which is in surface-contact with the front-surface portion (91b) of the flat tube (91), and the bracket (81), which is similar to that of the second embodiment. In an upper part of a back surface of the support plate (93), a sensor recessed portion (94), into which a part of the pressure sensor (42) is fitted, is formed.

In the third embodiment, as in the second embodiment, the spring (85) is interposed between the middle support portion (92) and the second middle case (32). The spring (85) urges the tube unit (90) and the second casing (30) in the direction such that the tube unit (90) and the second casing (30) become closer to each other, and the clothing item (2, 3) is clamped between the tube unit (90) and the second casing (30).

### Advantageous Effects of Third Embodiment

With the third embodiment, when a body motion of a subject acts on the back-surface portion (91a) of the flat tube (91) in the direction of an arrow in FIG. 17, the flat tube (91), which has flexibility, is squeezed in the front-back direction, and the internal pressure of the flat tube (91) changes. That is, with the third embodiment, because a body motion of a subject directly acts on the flat tube (91), the accuracy of biological information is reliably improved.

With the third embodiment, because the flat tube (91) is used as a part of the first member, the back case (21) of the first and second embodiments is not necessary, and the number of components can be reduced. The other advantageous effects are the same as those of first embodiment.

### Other Examples of Layout of Circuit Board In each of the embodiments described above, layout related the circuit board (50) may be any one of the following examples.

### First Example

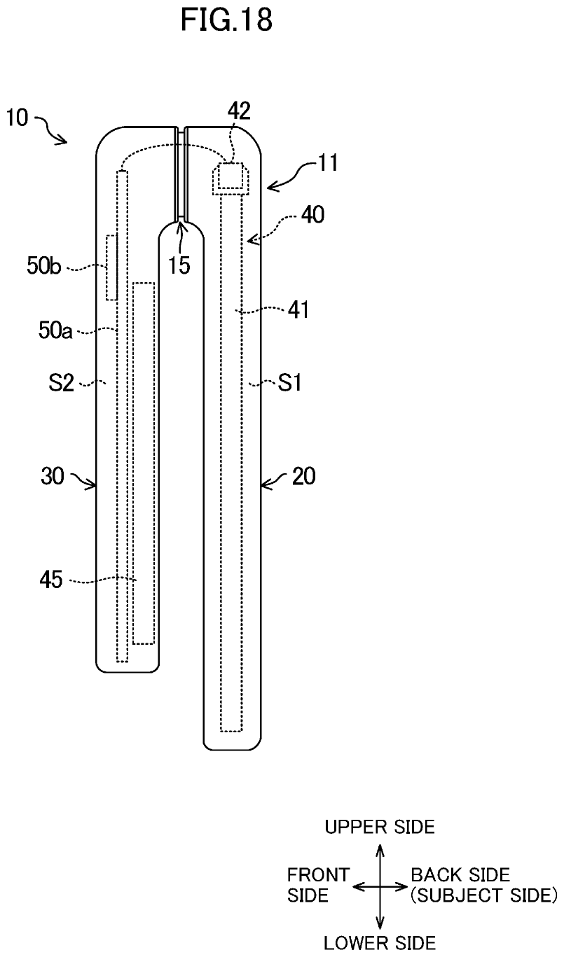

FIG. 18 illustrates a first example in which the circuit board (50) includes a main board (50a) and a communication board (50b). The main board (50a) is a signal processing circuit that processes a signal output from the pressure sensor (42), and a central processing unit (CPU), storage devices (such as a memory and a register), and the like are executed. The main board (50a) includes the body-motion-signal extracting unit (52), the heartbeat-signal extracting unit (53), the autonomic-nerve-information acquisition unit (54), the storage unit (55), and the like, which are described above. The communication board (50b) is a circuit for transmitting a signal processed by the main board (50a) to a predetermined wireless terminal (such as a smartphone, a tablet, or a PC) via wireless communication. That is, the communication board (50b) includes the communication unit (56) described above.

In the first example illustrated in FIG. 18, both of the main board (50a) and the communication board (50b) are contained in the second space (S2) of the second casing (30). For example, in the second space (S2), the rechargeable battery (45), the main board (50a), and the communication board (50b) are arranged in this order from the back side (subject side) toward the front side.

With the first example, because both of the main board (50a) and the communication board (50b) are not disposed in the first casing (20) on the subject side, the first casing (20) can be reduced in size and thickness. Thus, a sensation of tightness or an uncomfortable sensation experienced by a subject due to the presence of the first casing (20) can be suppressed.

### Second Example

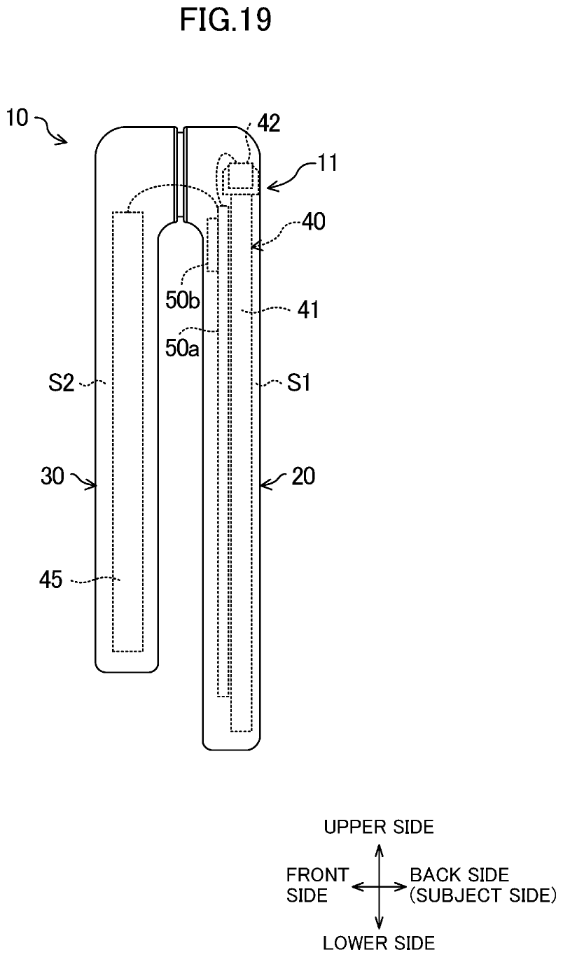

FIG. 19 illustrates a second example in which the circuit board (50) is disposed in the first space (S1) of the first casing (20). That is, both of the main board (50a) and the communication board (50b) are disposed in the first casing (20). In the first space (S1), the tube (41), the main board (50a), and the communication board (50b) are arranged in this order from the back side (subject side) toward the front side. On the other hand, the rechargeable battery (45) is disposed in the second space (S2) of the second casing (30). With this example, the second casing (30) can have a sufficient space for the rechargeable battery (45).

For example, the main board (50a) may be disposed in the second space (S2) of the second casing (30), and the communication board (50b) may be disposed in the first space (S1) of the first casing (20). The main board (50a) may be disposed in the first space (SI) of the first casing (20), and the communication board (50b) may be disposed in the second space (S2) of the second casing (30).

### Third Example

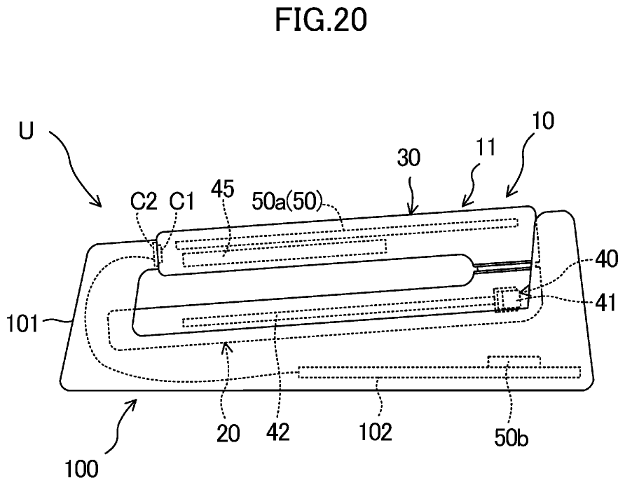

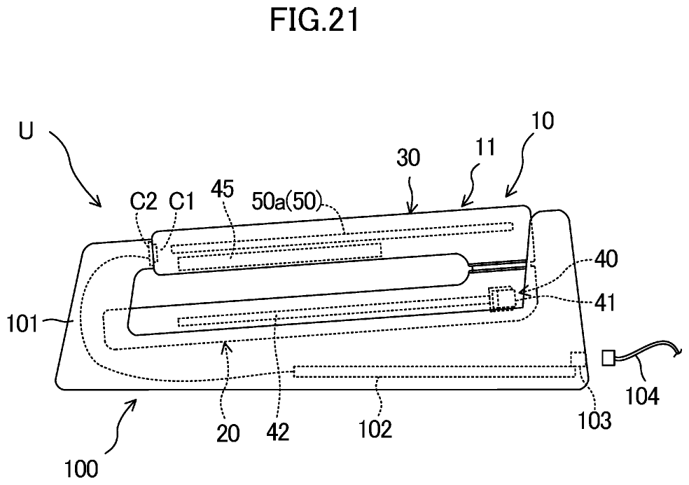

FIG. 20 illustrates a third example in which a unit (U) includes the biological information acquisition device (10) and a charging cradle (100). In the unit (U), the biological information acquisition device (10) can be placed on the upper side of the charging cradle (100). The second casing (30) of the biological information acquisition device (10) has a first terminal portion (C1). A case body (101) of the charging cradle (100) has a second terminal portion (C2).

A memory board (102), which is a storage unit, is disposed in the case body (101) of the charging cradle (100). In the third example, the communication board (50b) is disposed not in the biological information acquisition device (10) but in the case body (101) of the charging cradle (100).

As illustrated in FIG. 20, when the biological information acquisition device (10) is placed on the charging cradle (100), the contact points of the second terminal portion (C2) and the first terminal portion (C1) become connected. Thus, electric power is supplied from the charging cradle (100) to the rechargeable battery (45) of the biological information acquisition device (10), and the rechargeable battery (45) is charged. When the contact points of the first terminal portion (C1) and the second terminal portion (C2) are connected, data stored in the storage unit (55) of the biological information acquisition device (10) is transmitted to the memory board (102). Thus, data of signals related to a body motion of a subject is stored in the memory board (102). The data stored in the charging cradle (100) side is transmitted from the communication board (50b) to a wireless terminal via wireless communication.

### Fourth Example

FIG. 20 illustrates a fourth example in which the communication board (50b) is not disposed in the charging cradle (100), in contrast to the third example. In the charging cradle (100) of the fourth example, a connector (103) for outputting data stored in the memory board (102) to the outside via wired communication is disposed. A cable (104) such as a USB cable can be connected to the connector (103). Accordingly, data stored in the charging cradle (100) side is output to a predetermined terminal (a PC or the like) via the cable (104).

### Other Embodiments

An elastic member such as the clip (25) according to the first embodiment may be used in the second and third embodiments, and an elastic member such as the spring (85) according the second embodiment may be used in the first embodiment. That is, elements of the embodiments (including modifications) may be used in any combination as long as the basic function is not impaired.

In the third embodiment, the entirety of the first member may be a flat tube. In this case, because the first member includes only the flat tube (91), the number of components can be further reduced. In this case, the pressure sensor (42) may be disposed in the second casing (30).

In each of the embodiments, the LF/HF value is acquired as biological information. However, a body motion signal, a heartbeat signal, a breathing signal, or the like may be acquired as biological information. For example, information related to sleep of a subject may be acquired as biological information.

In each of the embodiments, the tube (41) or the flat tube (91) is used as a hollow member. However, the hollow member need not have a tubular shape, and may have, for example, a bag-like shape.

In each of the embodiments, at least one of a body motion signal, a heartbeat signal, and a breathing signal extracted by the circuit board (50) may be transmitted to a server on a network or to another communication terminal via the communication unit (56). In this case, the server or the communication terminal may acquire biological information (such as autonomic nerve information) based on these signals.

In each of the embodiments, the circuit board (50) is disposed in the second casing (30), which is a second member. However, the circuit board (50) may be disposed in the first casing (20), the connection member (15), or a member that is independent from the case unit (11).

The clamp member (11) in each of the embodiments is attached to the belt (2) and the trousers (3). However, the clamp member (11) may be attached to any clothing item worn by a subject, such as a skirt, a shoe, disposable underpants, or the like.

In each of the embodiments, a signal detected by the pressure sensor (42) may be directly output to an external terminal via the communication board (50b), and the terminal may perform signal processing.

### INDUSTRIAL APPLICABILITY

As described above, the present invention is useful as a biological information acquisition device.

### REFERENCE SIGNS LIST

2 belt (clothing item)

3 trousers (clothing item)

10 biological information acquisition device

11 case unit (clamp member)

15 connection member

20 first casing (first member)

25 clip (elastic member)

30 second casing (second member)

40 pressure sensing unit (body motion detector)

41 tube (hollow member, pressure sensitive portion)

42 pressure sensor

50 circuit board

61 first wall

62 second wall

65 support portion

74 peripheral wall

78 small-thickness portion

85 spring (elastic member)

90 tube unit (first member)

91 flat tube (hollow member, pressure sensitive portion)

1. A biological information acquisition device comprising:
a body motion detector that includes a pressure sensitive portion on which a body motion of a subject acts and that detects a signal indicating the body motion of the subject; and
a clamp member that clamps a clothing item of the subject,
wherein the body motion detector includes
a hollow member that is the pressure sensitive portion, and
a pressure sensor that detects a pressure of the hollow member as the signal,
wherein the clamp member includes
a first member that is disposed on the subject side of the clothing item, and
a second member that is disposed on an opposite side of the clothing item from the first member, and
wherein the pressure sensitive portion is disposed on the first member side.

2. The biological information acquisition device according to claim 1, comprising:
a circuit board that performs signal processing for acquiring biological information of the subject based on the signal detected by the body motion detector,
wherein the circuit board is not disposed on the first member side.

3. The biological information acquisition device according to claim 2,
wherein the circuit board is disposed on the second member side.

4. The biological information acquisition device according to claim 1, comprising:
an elastic member that urges the clamp member in such a way that the first member and the second member become closer to each other.

5. The biological information acquisition device according to claim 4,
wherein the clamp member includes a connection member that couples an end portion of the first member and an end portion of the second member to each other, and
wherein the elastic member is a plate spring that extends along the first member, the connection member, and the second member and that has a U-shaped thickness cross section.

6. The biological information acquisition device according to claim 1,
wherein the first member is a casing that contains the hollow member.

7. The biological information acquisition device according to claim 6,
wherein the casing includes
a first wall on which a body motion of the subject acts, and
a second wall that is disposed on an opposite side of the hollow member from the first wall, and
wherein a rigidity of the first wall is lower than a rigidity of the second wall.

8. The biological information acquisition device according to claim 6,
wherein the casing includes
a first wall on which a body motion of the subject acts,
a second wall that is disposed on an opposite side of the hollow member from the first wall, and
a peripheral wall that is disposed between the first wall and the second wall and that includes a small-thickness portion having a thickness smaller than a thickness of each of the first wall and the second wall.

9. The biological information acquisition device according to claim 6,
wherein the casing includes
a wall on which a body motion of the subject acts, and
a support portion that supports the wall in such a way that the wall is displaceable toward the hollow member.

10. The biological information acquisition device according to claim 1,
wherein at least a part of the first member also serves as the hollow member.

11. The biological information acquisition device according to claim 2, wherein the first member is a casing that contains the hollow member.

12. The biological information acquisition device according to claim 3, wherein the first member is a casing that contains the hollow member.

13. The biological information acquisition device according to claim 4, wherein the first member is a casing that contains the hollow member.

14. The biological information acquisition device according to claim 5, wherein the first member is a casing that contains the hollow member.

15. The biological information acquisition device according to claims 2, wherein at least a part of the first member also serves as the hollow member.

16. The biological information acquisition device according to claims 3, wherein at least a part of the first member also serves as the hollow member.

17. The biological information acquisition device according to claims 4, wherein at least a part of the first member also serves as the hollow member.

18. The biological information acquisition device according to claims 5, wherein at least a part of the first member also serves as the hollow member.
